(19) United States
(12) Patent Application Publication
(10) Pub. No.: US 2017/0000707 A1
(43) Pub. Date: Jan. 5, 2017

(54) Method for Dyeing Keratin Fibres

(71) Applicant: ALFA PARF GROUP S.p.A., Bologna (IT)

(72) Inventors: Katiuscia Grevalcuore, Bergamo (IT); Antonio Consoli, Urgnano (IT)

(21) Appl. No.: 15/193,840

(22) Filed: Jun. 27, 2016

(30) Foreign Application Priority Data
Jul. 2, 2015 (IT) 102015000030194

(51) Int. Cl. A61K 8/41; A61Q 5/10; A61K 8/49

(52) U.S. Cl. CPC A61K 8/411; A61K 8/4946; A61K 8/494; A61Q 5/10; A61K 2800/882; A61K 2800/4324

(57) ABSTRACT

Disclosed is a method of dyeing keratin fibres, in particular hair, which involves:
a) achieving the desired colour by mixing two or three compositions, each of which includes a single direct dye, magenta, blue or yellow, having similar performance characteristics
b) adding the mixture obtained at step a) to a diluent composition;
c) mixing and applying the composition obtained at step b) to the hair, optionally after checking the resulting colour by comparing it with reference standards.

The present invention relates to a cosmetic method for dyeing human keratin fibres, such as hair, by mixing the three primary colours together to obtain all types of shade. The present invention also relates to a composition which is either ready for immediate use with the aid of a dispensing device, or can be diluted in cosmetic treatments. The invention also involves the use of particular dispensing devices and systems.

### PRIOR ART

There are currently numerous trends in hair dyeing. In the past, the hair was mainly dyed to conceal areas of grey hair, whereas there is now increased demand for a hair colour based on fashion as an expression of the personality. Two hair-dyeing technologies are widely used. The first involves the use of oxidative dyes and an activator (oxidative hair dyes), while the second involves the use of direct dyes.

Oxidative dyes have become very important in the conventional hair dye field. The colour is created by reacting primary intermediates and couplers in the presence of an oxidant. Oxidative dyes give the best performance in terms of resistance to washing, and are therefore called permanent hair dyes. Direct dyes have become widely used in recent years and they can be used to create very bright tones. Particular combinations of Magenta, Blue and Yellow can be used to produce “fashion” shades (hair dyes with intense tone without level) like violet, red, purple and green, or natural shades like black, brown, gold and copper.

Direct dyes can be classified, according to their chemical charge, as anionic, cationic or non-ionic.

The hair colouring preparations that can be created with direct dyes can be classified, according to their ability to resist washing, as semipermanent or temporary. Semipermanent hair dyes can withstand up to 8 washes, and temporary dyes up to 2 washes (see table 1).

TABLE 1

| | TEMPORARY | SEMIPERMANENT | DEMIPERMANENT | PERMANENT |
|---|---|---|---|---|
| DURABILITY | 1-2 shampoos | 6-8 shampoos | 6-8 weeks | Permanent |
| PROPERTIES | | | | |
| LIGHTENING | No | No | No | Yes |
| EFFECT | | | | |
| COVERING | No | For people with a | For people with a | For people with |
| GREY HAIR | | maximum of 30% | maximum of 50% | 100% grey hair |
| | | grey hair (early | grey hair | |
| | | greying) | | |
| COLOUR | Only on | All tones with or | All tones with or | Lighter, identical |
| PROPERTIES | bleachedhair, | without bleaching | without bleaching | or darker tones |
| | or to add | | | With/without |
| | highlights | | | highlights |
| FINISHED | Ready to use | Ready to use | Mix before use: | Mix before use: |
| PRODUCT | | | 1 colouring product + | 1 colouring product + |
| | | | 1 to 3 parts activator. | 1 to 3 parts activator. |

Cationic or basic dyes have been widely used in recent years. These dyes bind to the keratin fibre much more efficiently than anionic or non-ionic dyes. Cationic dyes are more resistant to washing than the other two categories, and can therefore also be used to create semipermanent hair dyes.

Two particularly important cationic dyes, called Basic Red 51 and Basic Yellow 87 according to the INCI nomenclature (European Community Decision 2006/257/EC as amended—International Nomenclature of Cosmetic Ingredients), were introduced onto the cosmetic market in the 1990s. EP 0681464 reports the chemical structures of Basic Red 51 (BR51) and Basic Yellow 87 (BY87). Basic Red 51 and Basic Yellow 87 can be combined to give shades ranging from deep red to orange.

Basic Blue 124 (BB124) was introduced by BASF in 2013 (U.S. Pat. No. 8,268,014), thus completing the series of three cationic primary colours with which each type of shade (from natural to “fashion” colours) can be created. BB124 and BY87 can be combined in a formulation to obtain greens, BB124 and BR51 can be combined to obtain violets, and BB124, BY87 and BR51 can be combined to obtain colours ranging from black to chestnut to blonde with a natural tone.

To obtain greater brightness and evenness between roots and tips, BB 124, BR51 and BY87 can also be combined with a peroxide.

The hair dyes available in the hairdresser's salon consist of combinations of dyes defined as secondary colours, which are usually called shades. The International Colour Chart (ICC) is a system used to classify hair dyes. This means that each hair colouring composition has a code defining its colour result. Said code can be used by manufacturers of colour charts or hair dyes. In practice, the ICC system uses numbers to define the depth (level) and tone of a given colour.

The colour level indicates how light or dark the shade is. The ICC system assigns a number to measure the level of lightness or darkness. Said values range from 1 to 10, wherein 1 denotes the darkest shade (black) and 10 the lightest shade (lightest blonde). Some manufacturers also use the codes 11 and 12 on their hair dyeing products to indicate platinum blonde shades. The usual level numbers and names are as follows:

TABLE 2

| Level | Level name |
|---|---|
| 1 | Black |
| 2 | Very dark brown |
| 3 | Dark brown |
| 4 | Medium brown |
| 5 | Light brown |
| 6 | Dark blonde |
| 7 | Medium blonde |
| 8 | Light blonde |
| 9 | Very light blond |
| 10 | Lightest blond |
| 11 | Platinum blonde |

The tone indicates how cool or warm a colour is, and includes colours such as gold, ash and copper.

Although the level measurement is almost identical for all manufacturers, each manufacturer can vary the tone number at its discretion. Tone is indicated by a number, usually placed after the level, separated by a decimal point “.”, a comma “,” or a slash “/”. The classification used by the Applicant is set out below by way of example.

TABLE 3

| Tone number | Tone name |
|---|---|
| 0 | Natural (grey-neutral) |
| 1 | ash (blue) |
| 2 | Irisè (violet) |
| 3 | Gold (yellow) |
| 4 | Copper (orange) |
| 5 | Mahogany (violet red) |
| 6 | Red (red) |
| 7 | Matte (green) |
| 8 | Pearl |

Some hair colouring preparations can have a double tone, and it is usual to place two numbers after the decimal point of the level to express said characteristic. For example, if the colour chart contains the number 7.21, the first number indicates the medium blonde level (7), the second indicates the irisè tone (2), and the third number indicates a second ash tone (1). Said colour will be called “medium blonde irisè ash”.

Shades are created by the manufacturer by combining different components of yellow, red and blue, and secondary shades already created are available for use by the hairdresser, either alone or combined. This limits the creativity of the hairdresser, who has to use the secondary colour already created without being able to createcustomised shades.

Moreover the hairdresser has needs to add hair colour products to different hair treatments and cosmetic products to optimise the timing (2 in 1 service) or the results of the application. This means mixing the colouring products with hair reshaping treatments, other colour services, cleansing or care services, restructuring treatments or beauty treatments. The biggest problem is that not all dyeing technologies and hair colour preparations are versatile and usable (compatible and efficacious) in all other cosmetic preparations for the various hair treatments and expecially in all the pH values of use. The pH of the cosmetic product plays a strong role in determining compatibility and performance of the coloring preparation being added.

The packaging and tutorials may give instructions for an approximate dose to obtain various shades, but are not a reliable system reproducible in the salon to give the desired colour result.

The purpose of the invention is to solve said problems with a method involving the use of three solutions, each containing only one primary dye (Blue, Magenta or Yellow) to be suitably mixed in a diluent (clear) or added to any cosmetic form to apply to the hair in order to create any desired shade of colour.

### DESCRIPTION OF THE INVENTION

It has been found that said purposes can be achieved by a hair dyeing method involving:
a) Achieving the desired colour by mixing two or three solutions, each comprising one single direct dye, namely magenta, blue or yellow, having similar performance characteristics;
b) adding the mixture obtained at step a) to a diluent composition;
c) mixing and applying the composition obtained at step b) to the hair, optionally after checking the resulting colour by comparing it with reference standards;
d) the pH of the compositions thus obtained can range between 0.5 and 12, preferably between 1 and 11.

The invention also relates to kits containing two or three separate solutions, each of which contains only one primary direct dye; a diluent composition; devices designed for dispensing, measuring and mixing the primary dyes; optionally, instructions for creating the desired tones from the primary dyes, and reference tables for comparing colours.

### DETAILED DESCRIPTION OF THE INVENTION

The dyes which can be used according to the invention are cationic direct dyes having similar chemical/physical and performance characteristics in terms of binding to the hair and colour fading during washing. In particular, the dyes preferably used for blue are Basic Blue 124, HC Blue 16, HC Blue 17 and Basic Blue 75, but more preferably Basic Blue 124 and HC Blue 16; Basic Red 51 is preferably used for magenta, and the dyes preferably used for yellow are Basic Yellow 87 and Basic Yellow 57, more preferably Basic Yellow 87.

Other dyes that can be used are Basic Orange 31, Basic Red 76, and those described in WO 2014202152.

The dyes in solution can be dissolved in water and introduced into various cosmetic forms (liquid, gel, cream, milk, suspension or mousse) which can contain anionic, cationic, non-ionic or amphoteric surfactants; anionic, non-ionic or cationic gelling agents; fatty alcohols, fatty acids, alkyl derivatives such as straight, branched and cyclic paraffins; squalene and squalane; cyclosilicones, polydimethylsiloxanes, polyphenyldimethylsiloxanes, polyalkylsiloxanes, elastomers, acrylate/silicone copolymers, methyl trimethicone, polymethylsiloxanes, silicone rubbers, copolyol dimethicones, silicone resins and silicone powders.

The diluent composition comprises water, surfactants, preservatives and gelling agents.

The diluent can be a mask; a shampoo; an activator or a neutraliser with hydrogen peroxide; a hair colour composition; a semipermanent straightener, preferably based on alpha-keto acids or alpha-hydroxy acids; a hair-styling composition.

To minimise errors, the colour created by mixing two or three primary colours can be checked by visual comparison against a colour chart, for example printed on a plasticised backing, or with dedicated software for an application usable on a tablet, smartphone or PC (e.g. for Android®, iOS® or Windows®), using known algorithms.

The check can be conducted, for example, by applying a thin layer of the colour mixture to a transparent substrate overlaid on a coloured square previously printed or downloaded onto a smartphone, leaving one part uncovered, and comparing the colours, either visually or with a colorimeter, to see whether they fall within acceptable limits.

The dyeing results obtained from the examples below, expressed in L*a*b* values, were measured with a Minolta Chroma Meter CR-200 colorimeter.

In the L*a*b* colour space, L* indicates sheen and a* and b* are the colour coordinates. a* and b* indicate the colour directions: +a* is the direction of red, −a* is the direction of green, +b* is the direction of yellow and −b* is the direction of blue.

Differences in colour can be expressed by AE values, which are defined by the following equation:

ΔE=[(ΔL^{*})²+(Δa^{*})²+(Δb^{*})²]^{1/2}

In addition to the separate solutions, each of which contains only one primary direct dye and a diluent composition as defined above, the kit according to the invention can contain conventional devices suitable for dispensing, measuring and mixing the primary dyes. Examples of said devices include syringes, droppers, graduated bottles, tubes, vials, graduated cylinders and the like. The kit could also contain instructions for creating the desired tones from the primary dyes, and colour charts for comparing colours, either visually or with software and/or instrumentation.

### Examples

The formulations of the three colours used in the examples are listed below.

Formulation 1. BLUE PIGMENT
Formula A

| INCI | % |
|---|---|
| AQUA | Q.s. to 100 |
| PHENOXYETHANOL | 0.8 |
| POLYSORBATE 20 | 0.8 |
| BASIC BLUE 124 | 0.667 |
| ETHYLHEXYLGLYCERIN | 0.2 |

Formulation 2. MAGENTA PIGMENT
Formula B

| INCI | % |
|---|---|
| AQUA | Q.s. to 100 |
| PHENOXYETHANOL | 0.8 |
| POLYSORBATE 20 | 0.8 |
| BASIC RED 51 | 1.152 |
| ETHYLHEXYLGLYCERIN | 0.2 |

Formulation 3. YELLOW PIGMENT
Formula C

| INCI | % |
|---|---|
| AQUA | Q.s. to 100 |
| PHENOXYETHANOL | 0.8 |
| POLYSORBATE 20 | 0.8 |
| BASIC YELLOW 87 | 2.6151 |
| ETHYLHEXYLGLYCERIN | 0.2 |

Formulation 4. DILUENT
Formula D

| INCI | % |
|---|---|
| AQUA | Q.s. to 100 |
| PHENOXYETHANOL | 0.8 |
| POLYSORBATE 20 | 0.8 |
| HYDROXYPROPYL GUAR | 1.0 |
| HYDROXYPROPYLTRIMONIUM | |
| CHLORIDE | |
| ETHYLHEXYLGLYCERIN | 0.2 |

The following mixtures were created, showing the code of the shade created.

Formulation 5. BLACK 1.0

| PIGMENT | grams |
|---|---|
| BLUE | 3 |
| MAGENTA | 1 |
| YELLOW | 1 |
| DILUENT | 15 |

The L*a*b* values obtained are as follows:

| | |
|---|---|
| L | 20.44 |
| a | 0.39 |
| b | −1.59 |

Formulation 6. DEEP RED 8.65

| PIGMENT | grams |
|---|---|
| MAGENTA | 1 |
| YELLOW | 0.5 |
| DILUENT | 18.5 |

The L*a*b* values obtained are as follows:

| | |
|---|---|
| L | 31.33 |
| a | 33.81 |
| b | 15.05 |

Formulation 7. DEEP RED 8.66

| PIGMENT | grams |
|---|---|
| MAGENTA | 1 |
| YELLOW | 1 |
| DILUENT | 18 |

The L*a*b* values obtained are as follows:

| | |
|---|---|
| L | 33.2 |
| a | 37.12 |
| b | 21.69 |

Formulation 8. DEEP COPPER 8.46

| PIGMENT | grams |
|---|---|
| MAGENTA | 1 |
| YELLOW | 1.5 |
| DILUENT | 18 |

The L*a*b* values obtained are as follows:

| | |
|---|---|
| L | 36.18 |
| a | 29.41 |
| b | 24.23 |

Formulation 9. BROWN Shade 5.0

| PIGMENT | grams |
|---|---|
| BLUE | 0.8 |
| MAGENTA | 0.5 |
| YELLOW | 0.8 |
| DILUENT | 17.9 |

The L*a*b* values obtained are as follows:

| | |
|---|---|
| L | 29.43 |
| a | 4.23 |
| b | 6.36 |

Formulation 10. GOLD Shade 8.34

| PIGMENT | grams |
|---|---|
| MAGENTA | 0.04 |
| YELLOW | 6 |
| DILUENT | 18 |

The L*a*b* values obtained are as follows:

| | |
|---|---|
| L | 48.18 |
| a | 7.56 |
| b | 47.58 |

The shades reported were used in the following treatments:

1—Treatment designed to change hair shape with permanent technology,

2—Treatments designed to change hair shape with semipermanent technology,

3—Treatment designed to change hair shape with temporary technology,

4—Hair cleansing treatment,

5—Hair care, maintenance and beauty treatment,

6—Treatment designed to dye hair with a permanent or semipermanent hair dye,

7—Treatment designed to dye hair with the diluent composition,

8—Treatment with hydrogen peroxide and a heat source.

9—Styling treatment or hair finishing products. Treatment 1, designed to change the hair shape with permanent technology, can be further divided, depending on the active molecule used, into three different technologies, namely:

A—Thioglycolate

B—Guanidine

C—Soda

Method for Technology 1A:
a) Apply thioglycolate composition and leave to act for 30 minutes
b) Rinse with running water.
c) Create the desired shade by suitably combining Blue, Magenta and Yellow.
d) Add mixture created at point c) to the neutraliser in proportion to the desired depth of colour, and apply the resulting mixture to the hair for 10 minutes. In this specific case, the quantity of neutraliser used corresponds to the quantity of diluent shown in the table.
e) Rinse with running water.
f) Apply conditioner and leave to act for 5 minutes.
g) Dry hair.

The L*a*b* values obtained are as follows:

BLACK

| | |
|---|---|
| L | 14.68 |
| a | 0.69 |
| b | −0.72 |

DEEP RED

| | |
|---|---|
| L | 30.32 |
| a | 36.76 |
| b | 17.62 |

BROWN

| | |
|---|---|
| L | 19.78 |
| a | 2.86 |
| b | 2.23 |

Method for Technology 1B:
a) Mix formulation containing guanidine and the calcium hydroxide formula according to the manufacturer's instructions,
b) Apply mixture obtained at point c) to the hair and leave to act for 30 minutes,
c) Rinse with running water,
d) Apply the specific shampoo recommended by the manufacturer and rinse,
e) Repeat point d) twice more,
f) Create the desired shade by suitably combining Blue, Magenta and Yellow.
g) Add mixture created at point f) to the mask in proportion to the desired depth of colour, and apply the resulting mixture to the hair for 10 minutes. In this specific case, the quantity of conditioning mask used corresponds to the quantity of diluent shown in the table,
h) Rinse with running water,
i) Dry hair.

The L*a*b* values obtained are as follows:

BLACK

| | |
|---|---|
| L | 16.04 |
| a | 1.85 |
| b | 0.07 |

DEEP RED

| | |
|---|---|
| L | 28.9 |
| a | 39.68 |
| b | 20.27 |

BROWN

| | |
|---|---|
| L | 17.17 |
| a | 3.56 |
| b | 1.67 |

Method for Technology 1C:
a) Apply formula containing sodium hydroxide (or an alternative alkalizing agent such as potassium hydroxide) to the hair, comb hair and leave to act for 30 minutes,
b) Rinse with running water.
c) Apply the specific shampoo recommended by the manufacturer and rinse.
d) Create the desired shade by suitably combining Blue, Magenta and Yellow.
e) Add mixture created at point d) to mask in the quantity corresponding to the diluent in the tables. Leave to act for 6 minutes.
f) Rinse with running water.

g) Dry hair.

The L*a*b* values obtained are as follows:

BLACK

| | |
|---|---|
| L | 15.35 |
| a | 0.92 |
| b | −0.42 |

DEEP RED

| | |
|---|---|
| L | 30.42 |
| a | 36.25 |
| b | 17.58 |

BROWN

| | |
|---|---|
| L | 18.4 |
| a | 3.34 |
| b | 2.72 |

Treatment 2, designed to change the hair shape with semipermanent technology, consists of the following method:
a) Wash hair with shampoo recommended by the manufacturer.
b) Create the desired shade by suitably combining Blue, Magenta and Yellow.
c) Mix the glyoxylic-based straightener with the conditioning mixture obtained at point b) in proportion to the desired depth of colour, and apply to hair for 20 minutes. In this specific case the quantity of straightener was replaced with the same quantity of diluent as shown in the table.
d) Rinse and dry.
e) Run straightening iron over the hair 2 to 20 times, according to the manufacturer's instructions, at a temperature of up to 230° C.
f) Optionally rinse.

The L*a*b* values obtained are as follows:

BLACK

| | |
|---|---|
| L | 42.61 |
| a | −0.28 |
| b | 4.65 |

DEEP RED

| | |
|---|---|
| L | 55.78 |
| a | 38.02 |
| b | 26.58 |

BROWN

| | |
|---|---|
| L | 47.86 |
| a | 3.69 |
| b | 12.92 |

Treatment 3, designed to change the hair shape with temporary technology, consists of the following method:
a) Wash hair with shampoo
b) Create the desired shade by suitably combining Blue, Magenta and Yellow.
c) Apply straightening product based on cysteine, carbocysteine or cysteamine for 30 minutes.
d) Rinse with running water.
e) Apply formulation with hydrogen peroxide mixed with the mixture obtained at point b) and leave to act for 10 minutes. In this specific case the quantity of neutraliser was replaced with the same quantity of diluent as shown in the table.
f) Rinse with running water (do not use shampoo).
g) Dry hair.
h) Use straightening iron according to hair type, 10 times per lock of hair, at a temperature of up to 230° C.

The L*a*b* values obtained are as follows:

BLACK

| | |
|---|---|
| L | 31.52 |
| a | −1.4 |
| b | 3.56 |

DEEP RED

| | |
|---|---|
| L | 43.63 |
| a | 27.03 |
| b | 14.82 |

Hair cleansing treatment 4 consists of the following method:
a) Create the desired shade by suitably combining Blue, Magenta and Yellow.
b) Mix the mixture created at point a) with the shampoo in proportion to the desired depth of colour, and apply to the hair for 2 minutes. In this specific case the quantity of shampoo was replaced with the same quantity of diluent as shown in the table.
c) Rinse with running water.
d) Dry hair.

The L*a*b* values obtained are as follows:

BLACK

| | |
|---|---|
| L | 43.2 |
| a | −1.57 |
| b | −0.17 |

DEEP RED

| | |
|---|---|
| L | 54.14 |
| a | 13.55 |
| b | 11.67 |

BROWN

| | |
|---|---|
| L | 50.49 |
| a | 0.96 |
| b | 7.81 |

Hair care and beauty treatment 5 consists of the following method:
a) Create the desired shade by suitably combining Blue, Magenta and Yellow.
b) Mix the mixture created at point a) with the conditioner or mask in proportion to the desired depth of colour, and apply to the hair for 5 minutes. In this specific case the quantity of conditioner was replaced with the same quantity of diluent as shown in the table.
c) Rinse with running water.
d) Dry hair.

The L*a*b* values obtained are as follows:

BLACK

| | |
|---|---|
| L | 22.69 |
| a | 0.32 |
| b | 1.25 |

DEEP RED

| | |
|---|---|
| L | 36.26 |
| a | 32.9 |
| b | 21.24 |

BROWN

| | |
|---|---|
| L | 28.82 |
| a | 0.99 |
| b | 6.81 |

Treatment 6, designed to colour hair with a permanent or semipermanent dye, consists of the following method:
a) Create the desired shade by suitably combining Blue, Magenta and Yellow.
b) Mix oxidative dye with the composition of mixture a) in proportion to the desired depth of colour.
c) Mix the mixture obtained at point b) with a suitable activator in the proportion recommended by the manufacturer, and apply to hair. Leave to act for 30 minutes.
d) Rinse with running water.
e) Dry hair.

The data set out below were obtained with and without the composition of formulation 11, used as tone intensifier.

Formulation 11.

| | grams |
|---|---|
| MAGENTA | 5 |
| YELLOW | 5 |
| PERMANENT DYE | 50 |
| ACTIVATOR | 90 |

TABLE 4

| | L* | a* | b* | dL* | da* | db* | dE*ab |
|---|---|---|---|---|---|---|---|
| COLOUR RESULT | 17.39 | 3.99 | 4.07 | 1.34 | 2.27 | 2.16 | 3.41 |
| for Formulation 11 | | | | | | | |

Formulation 12.

| PIGMENT | grams |
|---|---|
| MAGENTA | 13.5 |
| YELLOW | 13.5 |
| SEMIPERMANENT DYE | 33 |
| ACTIVATOR | 120 |

TABLE 5

| | L* | a* | b* | dL* | da* | db* | dE*ab |
|---|---|---|---|---|---|---|---|
| COLOUR RESULT | 27.7 | 15.66 | 18.7 | 6.3 | 10.65 | 11.61 | 16.97 |
| for Formulation 12 | | | | | | | |

Treatment 8, with hydrogen peroxide and a heat source, consists of the following method:
a) Create the desired shade by suitably combining Blue, Magenta and Yellow,
b) Mix the mixture obtained at point a) with hydrogen peroxide in proportion to the desired depth of colour and apply to hair,
c) Apply a heat source to the hair, preferably with a straightening iron, up to 230° C., and run it over the hair at least 5 times,
d) Rinse with running water,
e) Dry hair.

Styling or hair finishing product treatment 9 consists of the following method:
a) Create the desired shade by suitably combining Blue, Magenta and Yellow,
b) Mix the mixture obtained at point a) with the styling product (gel, mousse, cream, liquid or paste) or the finishing product (oil, silicone composition, suspension or aqueous liquid) in proportion to the desired depth of colour, and apply to hair.

### Comparative Example

### Effect of pH on Performance

FORMULA E - not part of the invention

| INCI | % |
|---|---|
| AQUA | Q.s. to 100 |
| PHENOXYETHANOL | 0.8 |
| POLYSORBATE 20 | 0.8 |
| HC BLUE 15 | 0.3 |
| ETHYLHEXYLGLYCERIN | 0.2 |

Formula F

| INCI | % |
|---|---|
| AQUA | Q.s. to 100 |
| PHENOXYETHANOL | 0.8 |
| POLYSORBATE 20 | 0.8 |
| HC BLUE 16 | 0.3 |
| ETHYLHEXYLGLYCERIN | 0.2 |

Formula G

| INCI | % |
|---|---|
| AQUA | Q.s. to 100 |
| PHENOXYETHANOL | 0.8 |
| POLYSORBATE 20 | 0.8 |
| BASIC BLUE 124 | 0.3 |
| ETHYLHEXYLGLYCERIN | 0.2 |

Three black shades were created with BLUE PIGMENT containing HC BLUE 15 (FORMULA E), HC BLUE 16 (FORMULA F) and BASIC BLUE 124 (FORMULA G), and combined with the RED PIGMENT (FORMULA B) and YELLOW PIGMENT (FORMULA C) respectively. Comparison of formulations 13, 14 and 15 (BLACK 1.0)

| | BLACK with | BLACK with | BLACK with |
|---|---|---|---|
| | formulation | formulation | formulation |
| | 13 | 14 | 15 |
| PIGMENT | BLACK E | BLACK F | BLACK G |
| BLUE with formula E | 3 g | | |
| BLUE with formula F | | 3 g | |
| BLUE with formula G | | | 3 g |
| MAGENTA | 1 g | 1 g | 1 g |
| YELLOW | 1 g | 1 g | 1 g |
| DILUENT | 15 g | 15 g | 15 g |

The three black shades created with the three different blue pigments give a hair-lock colour with the following L a b values

TABLE 6

| | L* | a* | b* |
|---|---|---|---|
| BLACK formulation 13 | 27.8 | 15.66 | 16.5 |
| BLACK formulation 14 | 27.3 | 15.66 | 17.9 |
| BLACK formulation 15 | 27.7 | 15.66 | 18.7 |

The three black shades mixed with an acid treatment as for treatment 2, designed to change the hair shape with semipermanent technology, gave the following colour:

TABLE 7

| | Colour |
|---|---|
| BLACK formulation 13 | Blue-black |
| BLACK formulation 14 | Black |
| BLACK formulation 14 | Black |

The three black shades mixed with a basic treatment such as treatment 6, designed to colour hair with a permanent or semipermanent dye, gave the following colour:

TABLE 8

| | Colour |
|---|---|
| BLACK formulation 13 | Greenish-brown |
| BLACK formulation 14 | Black |
| BLACK formulation 15 | Black |

As will be seen from table 7 and table 8, the BLACK of formulation 13 undergoes a colour change if used at different pH values, whereas the BLACK of formulations 14 and 15 maintains the same tone at the two different pH values.

1. A method of dyeing keratin fibres, in particular hair, which comprises:
a) achieving the desired colour by mixing two or three solutions, each of which includes a single direct dye, magenta, blue or yellow having similar performance characteristics, with the proviso that the blue dye is not HC Blue 15;
b) adding the mixture obtained at step a) to a diluent composition;
c) mixing and application of the composition obtained at step b) to the hair, optionally after checking the resulting colour by comparison with a reference standard.

2. A method according to claim 1 wherein the magenta dye is Basic Red 51.

3. A method according to claim 1 wherein the yellow dye is selected from Basic Yellow 87 and Basic Yellow 57.

4. A method according to claim 1, wherein the blue dye is selected from Basic Blue 124, HC Blue 16, HC Blue 17 and Basic Blue 75.

5. A method according to claim 1 wherein the dye solutions optionally comprise anionic, cationic, non-ionic or amphoteric surfactants; anionic, non-ionic or cationic gelling agents; fatty alcohols, fatty acids, straight, branched or cyclic paraffins; squalene; squalane; cyclosilicones, polydimethylsiloxanes, polyphenyldimethylsiloxanes, polyalkylsiloxanes, elastomers, acrylate/silicone copolymers, methyl trimethicone, polymethylsiloxanes, silicone rubbers, dimethicone copolyols, silicone resins and silicone powders.

6. A method according to claim 5 wherein the dye in solution is formulated as a gel, cream, milk, suspension or mousse.

7. A method according to claim 1 wherein the diluent composition comprises water, surfactants, preservatives and gelling agents.

8. A method according to claim 1 where in the diluent is a mask; an activator or a neutralising agent comprising hydrogen peroxide; a hair colour composition ; a semipermanent straightening agent; or a hair styling composition.

9. A kit comprising two or three separate compositions, each of which contains solely a direct primary dye as defined in claim 1; a diluent composition comprising water, surfactants, preservatives and gelling agents; devices suitable for dispensing, measuring and mixing the primary dyes; and optionally instructions for obtaining the desired tones starting from primary dyes and colour charts for the comparison of colour results.

10. The method according to claim 4, wherein the blue dye is Basic Blue 124 or HC Blue 16.

11. The method according to claim 8, wherein the semipermanent straightening agent is based on alpha-ketoacids or alpha-hydroxy acids.
